(19) United States
(12) Patent Application Publication
(10) Pub. No.: US 2013/0116342 A1
(43) Pub. Date: May 9, 2013

(54) SALT-RESISTANT EMULSIONS

(71) Applicant: Annette Faith Dexter, Salisbury (AU)

(72) Inventor: Annette Faith Dexter, Salisbury (AU)

(73) Assignee: PEPFACTANTS PTY LTD, St Lucia, Queensland (AU)

(21) Appl. No.: 13/636,185

(22) PCT Filed: Mar. 22, 2011
(86) PCT No.: PCT/AU11/00319

§ 371 Date: Sep. 20, 2012

(30) Foreign Application Priority Data
Mar. 22, 2010 (AU) 2010901217

(51) Int. Cl. C07K 14/00

(52) U.S. Cl. CPC C07K 14/001
USPC 514/773; 530/326; 507/241; 507/244; 426/330.6; 426/602

(57) ABSTRACT

The invention relates to methods of modulating the stability of emulsions, especially the stability of emulsions to flocculation and coalescence. The use of peptide emulsifiers comprising at least one side chain carboxylate group in preparing emulsions that are stable to flocculation or coalescence in the presence of salt is also described.

### FIELD OF THE INVENTION

The invention generally relates to methods of modulating the stability of emulsions, in particular modulating the stability of emulsions to flocculation and coalescence. More particularly, the invention relates to use of peptide emulsifiers containing carboxylate groups for the preparation of emulsions that are resistant to flocculation or coalescence in the presence of salt. Utility of the methods and peptides for use in the method are also described.

### BACKGROUND OF THE INVENTION

Surface-active agents (surfactants) are chemical species that are able to adsorb at fluid-fluid interfaces to reduce the interfacial tension. They are used in the preparation of oil and water emulsions for a wide range of applications, for example oil recovery, drilling, metalworking, lubrication, catalysis, cleaning, agrichemical dispersions, drug delivery, processed foods and personal care. Surfactants may be nonionic or ionic, with ionic surfactants further subdivided into anionic, cationic and zwitterionic (amphoteric) classes. Surfactants can be used to facilitate the formation of emulsions and increase the lifetime of an emulsion once formed.

Emulsions are thermodynamically unstable suspensions or dispersions of one liquid in a second liquid with which it is not miscible. Commonly one of the liquids is water, and the second, immiscible liquid is referred to as an oil. The lifetime of an emulsion is increased by mechanisms that inhibit the various known modes of emulsion instability, including creaming, Ostwald ripening, flocculation and coalescence. For the purposes of this invention, the mechanisms of primary importance are flocculation, the process by which two or more drops collide and adhere to one another while maintaining separate entities and coalescence, the process by which two or more drops collide and join to form a single drop.

Emulsions are generally unstable in the presence of salt, particularly high concentrations of salt (Binks 1998). There is a need for methods of increasing the stability of emulsions to the addition of salt and/or allowing an emulsion to be initially prepared in the presence of salt.

The present inventor has surprisingly found that the preparation of emulsions resistant to flocculation or coalescence in the presence of salt can be facilitated by the use of peptide emulsifiers containing carboxylate groups.

### SUMMARY OF THE INVENTION

The present invention is predicated in part on the determination that emulsions may be resistant to flocculation or coalescence in the presence of salt if the emulsifier used to prepare the emulsion contains a strongly hydrated ionic group, such as a carboxylate group.

In one aspect the present invention provides a method of stabilizing an emulsion against flocculation or coalescence, said method comprising forming the emulsion in the presence of at least one peptide emulsifier, said at least one peptide emulsifier comprising one or more amino acid residues with a side chain carboxylate group.

The emulsions formed by the method of the invention are resistant to flocculation or coalescence in the presence of salt. In some embodiments, the salt is present in the bulk water phase at the time the emulsion is formed. In some embodiments, the salt is added to the emulsion after formation.

In some embodiments, the peptide emulsifier is an α-helical peptide. In other embodiments, the peptide emulsifier is a β-strand peptide. In some embodiments the peptide emulsifier has more than one amino acid residue with a side chain carboxylic acid group. In some embodiments, the peptide emulsifier comprises one or more amino acid residues with side chains bearing other charged non-carboxylic acid groups. In some embodiments, the peptide emulsifier has a net negative charge, a net positive charge or is zwitterionic. In some embodiments, the emulsion is formed in the presence of a mixture of peptide emulsifiers, at least one of which comprises one or more amino acid residues with a side chain carboxylate group.

In another aspect of the invention there is provided a method of making an emulsion resistant to flocculation or coalescence comprising:
mixing an aqueous phase and an oil phase together in the presence of at least one peptide emulsifier comprising one or more amino acid residues with a side chain carboxylate group.

In some embodiments, the peptide emulsifier is solubilized in the aqueous phase before mixing. In some embodiments, the one or more side chain carboxylate groups are oriented around the liquid-liquid interface in the emulsion. In some embodiments, the peptide emulsifier is a mixture of peptide emulsifiers, at least one of which comprises one or more amino acid residues with a side chain carboxylate group.

In yet another aspect, the present invention provides a salt-resistant emulsion comprising at least one peptide emulsifier comprising one or more amino acid residues with a side chain carboxylate group.

In some embodiments, the peptide emulsifier is an α-helical peptide. In some embodiments, the peptide emulsifier is a β-strand peptide. In some embodiments, the one or more carboxylate groups of the peptide emulsifier are oriented around the liquid-liquid interface of the emulsion. In some embodiments, the peptide emulsifier is a mixture of peptide emulsifiers, at least one of which comprises one or more amino acid residues with a side chain carboxylate group.

In yet a further aspect, the present invention provides a peptide having one of the following sequences:

SEQ ID NO: 2
Ac-LEELADS LEELAEQ VEELLSA-CONH₂
SEQ ID NO: 3
Ac-EISALEA EISALEA EISALEA-CONH₂
SEQ ID NO: 4
Ac-AISELEA EISALEA EIESLAA-CONH₂
SEQ ID NO: 5
Ac-AIESLAE SIEELAE AISELAA-CONH₂
SEQ ID NO: 6
H₂N-P LAEIADS LAEIAEQ VAELIEA VED-CO₂H
SEQ ID NO: 7
H₂N-P LEAIADS LEAIAEQ VEALIEA VAD-CO₂H
SEQ ID NO: 8
H₂N-PG IAELEAE LSAVAEA LEAILAE LD-CO₂H
SEQ ID NO: 9
Ac-LAELESL LAELEAL VAELLSA-CONH₂.

### DESCRIPTION OF THE INVENTION

### Definitions

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integers or steps.

The singular forms “a”, “an” and “the” include plural aspects unless the context clearly dictates otherwise.

As used herein, the term “emulsion” refers to a suspension or dispersion of a first liquid suspended or dispersed in a second liquid in which the first liquid is poorly soluble or non-miscible. The first liquid is referred to as the dispersed phase and the second liquid is referred to as the continuous phase. The dispersed phase may form droplets which are dispersed throughout the continuous phase in a heterogeneous or homogeneous manner. Illustrative examples of emulsions include oil-in-water emulsions in which the oil forms the dispersed phase and the water forms the continuous phase, and water-in-oil emulsions in which the water forms the dispersed phase and the oil forms the continuous phase. In addition, “multiple emulsions” may be formed in which droplets of a first discontinuous phase contain smaller droplets of a second discontinuous phase, which may or may not be similar in composition to the continuous phase containing the first discontinuous phase. Illustrative examples of multiple emulsions include water-in-oil-in-water emulsions in which oil forms the first discontinuous phase and water forms the second discontinuous phase, and oil-in-water-in-oil emulsions in which the water forms the first discontinuous phase and oil forms the second discontinuous phase.

The term “emulsifier” as used herein refers to a compound which is capable of stabilizing an emulsion by increasing its kinetic stability. In the present invention the emulsifier is a peptide surfactant or mixture of peptide surfactants.

As used herein, the term “surfactant” refers to a chemical agent capable of lowering the interfacial tension at a liquid-liquid interface, for example by adsorbing at the liquid-liquid interface. Surfactants have a polar moiety or region and a non-polar moiety or region providing an affinity for the liquid-liquid interface. In the case of peptide surfactants, neutral polar moieties include backbone amide groups, serine, threonine, cysteine, asparagine and glutamine side chains, while charged polar moieties include aspartate, glutamate and C-terminal anions and histidine, lysine, arginine and N-terminal cations. Examples of non-polar moieties in peptides include alanine, valine, leucine, isoleucine, tyrosine, tryptophan and phenylalanine side chains. Anionic, cationic or zwitterionic peptide surfactant may be designed using various combinations of these sidechains arrayed along a peptide amide backbone.

As used herein, the term “interfacial tension” refers to the energy of a liquid-liquid interface, and quantitatively describes the tendency of a liquid to minimize its interfacial area Examples of methods used to determine interfacial tension include, but are not limited to, maximum bubble pressure, axisymmetric drop or bubble shape, du Nouy ring, Wilhelmy plate, spinning drop and drop weight methods. Interfacial tension is commonly reported in units of mN m⁻¹, numerically equivalent to dynes cm⁻¹.

As used herein, the term “liquid-liquid interface” refers to a surface forming the common boundary between two adjacent non-miscible liquids, such as oil and water. In some cases, a liquid-liquid interface may also be referred to more simply as a fluid interface.

As used herein the term “affinity for the liquid-liquid interface” means that emulsifiers from a bulk solution are attracted to or adsorbed at the liquid-liquid interface such that the concentration of emulsifier at the liquid-liquid interface is greater than the concentration of emulsifier in the bulk solution. In general, emulsifiers, including peptide emulsifiers, have hydrophobic and hydrophilic regions and align themselves at the interface to minimize their free energy on adsorption, typically such that the hydrophobic moiety or moieties are in contact with a non-polar portion of the interface and their hydrophilic moiety or moieties are in contact with a polar portion of the interface. With peptide emulsifiers, hydrophobic moieties that are non-adjacent in the peptide sequence may come to lie close to each other at an interface as a result of folding of the peptide chain, for example into an α-helical structure. For example, using suitable design approaches, hydrophobic moieties that are non-adjacent in the peptide sequence can be induced to form a single hydrophobic face to an α-helical structure on folding at an interface. Similarly, hydrophilic moieties that are non-adjacent in the peptide sequence may come to lie close to each other at an interface as a result of folding of the peptide chain.

As used herein, the term “peptide” refers to two or more naturally occurring or non-naturally occurring amino acids joined by peptide bonds. Generally, peptides will range from about 2 to about 80 amino acid residues in length, usually from about 5 to about 60 amino acid residues in length and more usually from about 10 to about 40 amino acid residues in length. The peptide may also be a retro-inverso peptide. The peptide may contain α-amino acid residues, β-amino acid residues, D-amino acid residues, L-amino acid residues, naturally occurring amino acid residues or non-naturally occurring amino acid residues.

The amino acid may also be further substituted in the α-position or the β-position with a group selected from —C₁-C₆alkyl, —(CH₂)ₙCOR₁, —(CH₂)ₙR₂, —PO₃H, —(CH₂)ₙheterocyclyl or —(CH₂)ₙaryl where R₁ is —OH, —NH₂, —NHC₁-C₃alkyl, —OC₁-C₃alkyl or —C₁-C₃alkyl and R₂ is —OH, —SH, —SC₁-C₃alkyl, —OC₁-C₃alkyl, —C₃-C₁₂cycloalkyl, —NH₂, —NHC₁-C₃alkyl or —NHC(C═NH)NH₂ and where each alkyl, cycloalkyl, aryl or heterocyclyl group may be substituted with one or more groups selected from —OH, —NH₂, —NHC₁-C₃alkyl, C₁-C₃alkyl, —SH, —SC₁-C₃alkyl, —CO₂H, —CO₂C₁-C₃alkyl, —CONH₂ or —CONHC₁-C₃alkyl.

Amino acid structure and single and three letter abbreviations used throughout the specification are defined in Table 1, which lists the twenty naturally occurring amino acids which occur in proteins as L-isomers.

TABLE 1
(1)

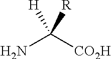

(2)

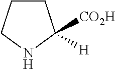

| | Three-letter | One-letter | Structure of side chain |
|---|---|---|---|
| Amino Acid | Abbreviation | symbol | (R) |
| Alanine | Ala | A | —CH₃ |
| Arginine | Arg | R | —(CH₂)₃NHC(═N)NH₂ |
| Asparagine | Asn | N | —CH₂CONH₂ |
| Aspartic acid | Asp | D | —CH₂CO₂H |
| Cysteine | Cys | C | —CH₂SH |
| Glutamine | Gln | Q | —(CH₂)₂CONH₂ |
| Glutamic acid | Glu | E | —(CH₂)₂CO₂H |
| Glycine | Gly | G | —H |
| Histidine | His | H | —CH₂(4-imidazolyl) |
| Isoleucine | Ile | I | —CH(CH₃)CH₂CH₃ |
| Leucine | Leu | L | —CH₂CH(CH₃)₂ |
| Lysine | Lys | K | —(CH₂)₄NH₂ |
| Methionine | Met | M | —(CH₂)₂SCH₃ |
| Phenylalanine | Phe | F | —CH₂Ph |
| Proline | Pro | P | see formula (2) above for |
| | | | structure of amino acid |
| Serine | Ser | S | —CH₂OH |
| Threonine | Thr | T | —CH(CH₃)OH |
| Tryptophan | Trp | W | —CH₂(3-indolyl) |
| Tyrosine | Tyr | Y | —CH₂(4-hydroxyphenyl) |
| Valine | Val | V | —CH(CH₃)₂ |

The term “α-amino acid” as used herein, refers to a compound having an amino group and a carboxyl group in which the amino group and the carboxyl group are separated by a single carbon atom, the α-carbon atom. An α-amino acid includes naturally occurring and non-naturally occurring L-amino acids and their D-isomers and derivatives thereof such as salts or derivatives where functional groups are protected by suitable protecting groups. The α-amino acid may also be further substituted in the α-position with a group selected from —C₁-C₆alkyl, —(CH₂)ₙCOR₁, —(CH₂)ₙR₂, —PO₃H, —(CH₂)ₙheterocyclyl or —(CH₂)ₙaryl where R₁ is —OH, —NH₂, —NHC₁-C₃alkyl, —OC₁-C₃alkyl or —C₁-C₃alkyl and R₂ is —OH, —SH, —SC₁-C₃alkyl, —OC₁-C₃alkyl, —C₃-C₁₂cycloalkyl, —NH₂, —NHC₁-C₃alkyl or —NHC(C═NH)NH₂ and where each alkyl, cycloalkyl, aryl or heterocyclyl group may be substituted with one or more groups selected from —OH, —NH₂, —NHC₁-C₃allyl, —OC₁-C₃alkyl, —SH, —SC₁-C₃alkyl, —CO₂H, —CO₂C₁-C₃alkyl, —CONH₂ or —CONHCᵣ—C₃alkyl.

As used herein, the term “β-amino acid” refers to an amino acid that differs from an α-amino acid in that there are two (2) carbon atoms separating the carboxyl terminus and the amino terminus. As such, β-amino acids with a specific side chain can exist as the R or S enantiomers at either of the α (C2) carbon or the β (C3) carbon, resulting in a total of 4 possible isomers for any given side chain. The side chains may be the same as those of naturally occurring α-amino acids (see Table 1 above) or may be the side chains of non-naturally occurring amino acids (see Table 2 below).

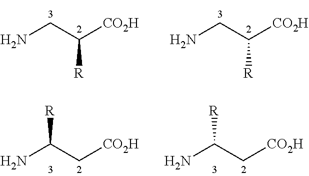

Furthermore, the β-amino acids may have mono-, di-, tri- or tetra-substitution at the C2 and C3 carbon atoms. Mono-substitution may be at the C2 or C3 carbon atom. Di-substitution includes two substituents at the C2 carbon atom, two substituents at the C3 carbon atom or one substituent at each of the C2 and C3 carbon atoms. Tri-substitution includes two substituents at the C2 carbon atom and one substituent at the C3 carbon atom or two substituents at the C3 carbon atom and one substituent at the C2 carbon atom. Tetra-substitution provides for two substituents at the C2 carbon atom and two substituents at the C3 carbon atom. Suitable substituents include —C₁-C₆alkyl, —(CH₂)ₙCOR₁, —(CH₂)ₙR₂, —PO₃H, —(CH₂)ₙheterocyclyl or —(CH₂)ₙaryl where R₁ is —OH, —NH₂, —NHC₁-C₃alkyl, —OC₁-C₃alkyl or —C₁-C₃alkyl and R₂ is —OH, —SH, —SC₁-C₃alkyl, —OC₁-C₃alkyl, —C₃-C₁₂cycloalkyl, —NH₂, —NHC₁-C₃alkyl or —NHC(C═NH)NH₂ and where each alkyl, cycloalkyl, aryl or heterocyclyl group may be substituted with one or more groups selected from —OH, —NH₂, —NHC₁-C₃alkyl, —OC₁-C₃alkyl, —SH, —SC₁-C₃alkyl, —CO₂H, —CO₂C₁-C₃alkyl, —CONH₂ or —CONHC₁-C₃alkyl.

Other suitable β-amino acids include conformationally constrained β-amino acids. Cyclic β-amino acids are conformationally constrained and are generally not accessible to enzymatic degradation. Suitable cyclic β-amino acids include, but are not limited to, cis- and trans-2-aminocyclopropyl carboxylic acids, 2-aminocyclobutyl and cyclobutenyl carboxylic acids, 2-aminocyclopentyl and cyclopentenyl carboxylic acids, 2-aminocyclohexyl and cyclohexenyl carboxylic acids and 2-amino-norbornane carboxylic acids and their derivatives, some of which are shown below:

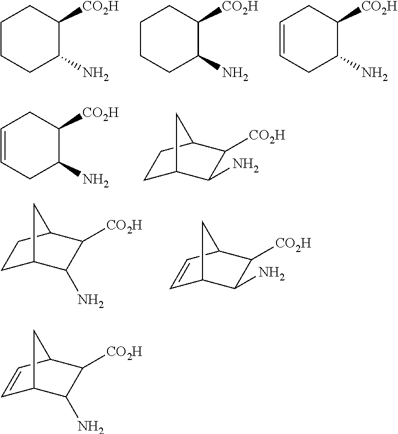

Suitable derivatives of β-amino acids include salts and may have functional groups protected by suitable protecting groups.

The term “non-naturally occurring amino acid” as used herein, refers to amino acids having a side chain that does not occur in the naturally occurring L-α-amino acids listed in Table 1. Examples of non-natural amino acids and derivatives include, but are not limited to, use of norleucine, 4-amino butyric acid, 4-amino-3-hydroxy-5-phenylpentanoic acid, 6-aminohexanoic acid, t-butylglycine, norvaline, phenylglycine, ornithine, citrulline, sarcosine, 4-amino-3-hydroxy-6-methylheptanoic acid, 2-thienyl alanine and/or D-isomers of amino acids. A list of unnatural amino acids that may be useful herein is shown in Table 2.

TABLE 2

| Non-conventional | | Non-conventional | |
|---|---|---|---|
| amino acid | Code | amino acid | Code |
| α-aminobutyric acid | Abu | L-N-methylalanine | Nmala |
| α-amino-α-methylbutyrate | Mgabu | L-N-methylarginine | Nmarg |
| aminocyclopropane- | Cpro | L-N-methylasparagine | Nmasn |
| carboxylate | | L-N-methylaspartic acid | Nmasp |
| aminoisobutyric acid | Aib | L-N-methylcysteine | Nmcys |
| aminonorbornyl- | Norb | L-N-methylglutamine | Nmgln |
| carboxylate | | L-N-methylglutamic acid | Nmglu |
| cyclohexylalanine | Chexa | L-N-methylhistidine | Nmhis |
| cyclopentylalanine | Cpen | L-N-methylisolleucine | Nmile |
| D-alanine | Dal | L-N-methylleucine | Nmleu |
| D-arginine | Darg | L-N-methyllysine | Nmlys |
| D-aspartic acid | Dasp | L-N-methylmethionine | Nmmet |
| D-cysteine | Dcys | L-N-methylnorleucine | Nmnle |
| D-glutamine | Dgln | L-N-methylnorvaline | Nmnva |
| D-glutamic acid | Dglu | L-N-methylornithine | Nmorn |
| D-histidine | Dhis | L-N-methylphenylalanine | Nmphe |
| D-isoleucine | Dile | L-N-methylproline | Nmpro |
| D-leucine | Dleu | L-N-methylserine | Nmser |
| D-lysine | Dlys | L-N-methylthreonine | Nmthr |
| D-methionine | Dmet | L-N-methyltryptophan | Nmtrp |
| D-ornithine | Dorn | L-N-methyltyrosine | Nmtyr |
| D-phenylalanine | Dphe | L-N-methylvaline | Nmval |
| D-proline | Dpro | L-N-methylethylglycine | Nmetg |
| D-serine | Dser | L-N-methyl-t-butylglycine | Nmtbug |
| D-threonine | Dthr | L-norleucine | Nle |
| D-tryptophan | Dtrp | L-norvaline | Nva |
| D-tyrosine | Dtyr | α-methyl-aminoisobutyrate | Maib |
| D-valine | Dval | α-methyl-aminobutyrate | Mgabu |
| D-α-methylalanine | Dmala | α-methylcyclohexylalanine | Mchexa |
| D-α-methylarginine | Dmarg | α-methylcyclopentylalanine | Mcpen |
| D-α-methylasparagine | Dmasn | α-methyl-α-napthylalanine | Manap |
| D-α-methylaspartate | Dmasp | α-methylpenicillamine | Mpen |
| D-α-methylcysteine | Dmcys | N-(4-aminobutyl)glycine | Nglu |
| D-α-methylglutamine | Dmgln | N-(2-aminoethyl)glycine | Naeg |
| D-α-methylhistidine | Dmhis | N-(3-aminopropyl)glycine | Norn |
| D-α-methylisoleucine | Dmile | N-amino-α-methylbutyrate | Nmaabu |
| D-α-methylleucine | Dmleu | α-napthylalanine | Anap |
| D-α-methyllysine | Dmlys | N-benzylglycine | Nphe |
| D-α-methylmethionine | Dmmet | N-(2-carbamylethyl)glycine | Ngln |
| D-α-methylornithine | Dmorn | N-(carbamylmethyl)glycine | Nasn |
| D-α-methylphenylalanine | Dmphe | N-(2-carboxyethyl)glycine | Nglu |
| D-α-methylproline | Dmpro | N-(carboxymethyl)glycine | Nasp |
| D-α-methylserine | Dmser | N-cyclobutylglycine | Ncbut |
| D-α-methylthreonine | Dmthr | N-cycloheptylglycine | Nchep |
| D-α-methyltryptophan | Dmtrp | N-cyclohexylglycine | Nchex |
| D-α-methyltyrosine | Dmty | N-cyclodecylglycine | Ncdec |
| D-α-methylvaline | Dmval | N-cylcododecylglycine | Ncdod |
| D-N-methylalanine | Dnmala | N-cyclooctylglycine | Ncoct |
| D-N-methylarginine | Dnmarg | N-cyclopropylglycine | Ncpro |
| D-N-methylasparagine | Dnmasn | N-cycloundecylglycine | Ncund |
| D-N-methylasparatate | Dnmasp | N-(2,2-diphenylethyl)glycine | Nbhm |
| D-N-methylcysteine | Dnmcys | N-(3,3-diphenylpropyl)glycine | Nbhe |
| D-N-methylglutamine | Dnmgln | N-(3-guanidinopropyl)glycine | Narg |
| D-N-methylglutamate | Dnmglu | N-(1-hydroxyethyl)glycine | Nthr |
| D-N-methylhistidine | Dnmhis | N-(hydroxyethyl))glycine | Nser |
| D-N-methylisoleucine | Dnmile | N-(imidazolylethyl))glycine | Nhis |
| D-N-methylleucine | Dnmleu | N-(3-indolylyethyl)glycine | Nhtrp |
| D-N-methyllysine | Dnmlys | N-methyl-γ-aminobutyrate | Nmgabu |
| N-methylcyclohexylalanine | Nmchexa | D-N-methylmethionine | Dnmmet |
| D-N-methylorinithine | Dnmorn | N-methylcyclopentylalanine | Nmcpen |
| N-methylglycine | Nala | D-N-methylphenylalanine | Dnmphe |
| N-methylaminoisobutyrate | Nmaib | D-N-methylproline | Dnmpro |
| N-(1-methylpropyl)glycine | Nile | D-N-methylserine | Dnmser |
| N-(2-methylpropyl)glycine | Nleu | D-N-methylthreonine | Dnmthr |
| D-N-methyltryptophan | Dnmtrp | N-(1-methylethyl)glycine | Nval |
| D-N-methyltyrosine | Dnmtyr | N-methylnapthylalanine | Nmanap |
| D-N-methylvaline | Dnmval | N-methylpenicillamine | Nmpen |
| γ-aminobutyric acid | Gabu | N-(p-hydroxyphenyl)glycine | Nhtyr |
| L-t-butylglycine | Tbug | N-(thiomethyl)glycine | Ncys |
| L-ethylglycine | Etg | penicillamine | Pen |
| L-homophenylalanine | Hphe | L-α-methylalanine | Mala |
| L-α-methylarginine | Marg | L-α-methylasparagine | Masn |
| L-α-methylasparate | Masp | L-α-methyl-t-butylglycine | Mtbug |
| L-α-methylcysteine | Mcys | L-methylethylglycine | Metg |
| L-α-methylglutamine | Mgln | L-α-methylglutamate | Mglu |
| L-α-methylhistidine | Mhis | L-α-methylhomophenylalanine | Mhphe |
| L-α-methylisoleucine | Mile | N-(2-methylthioethyl)glycine | Nmet |
| L-α-methylleucine | Mleu | L-α-methyllysine | Mlys |
| L-α-methylmethionine | Mmet | L-α-methylnorleucine | Mnle |
| L-α-methylnorvaline | Mnva | L-α-methylornithine | Morn |
| L-α-methylphenylalanine | Mphe | L-α-methylproline | Mpro |
| L-α-methylserine | Mser | L-α-methylthreonine | Mthr |
| L-α-methyltryptophan | Mtrp | L-α-methyltyrosine | Mtyr |
| L-α-methylvaline | Mval | L-N-methylhomophenylalanine | Nmhphe |
| N-(N-(2,2-diphenylethyl) | Nnbhm | N-(N-(3,3-diphenylpropyl) | Nnbhe |
| carbamylmethyl)glycine | | carbamylmethyl)glycine | |
| 1-carboxy-1-(2,2- | Nmbc | | |
| diphenylethylamino)cyclopropane | | | |

As used herein, the term “amino acid residue with a side chain carboxylate group” refers to an α-, β-, L- or D-amino acid which has a side chain bearing a carboxylate ion. Examples of such amino acid residues include L-glutamic acid, D-glutamic acid, L-aspartic acid, D-aspartic acid, L-α-methylglutamic acid, D-α-methylglutamic acid, L-α-methylaspartic acid, D-α-methylaspartic acid, L-homoglutamic acid and D-homoglutamic acid.

The term “oriented around the liquid-liquid interface” refers to amino acid side chains, including those having carboxylate groups, being located in a close proximity and laterally to the liquid-liquid interface rather than being located within the bulk oil or aqueous phases. For example, in FIG. 1, if the liquid-liquid interface bisects the peptide horizontally and centrally (as marked by 14 and 5), the side chains oriented around the liquid-liquid interface are at positions 3, 14, 7, 12, 5 and 16.

The term “alkyl” as used herein refers to straight chain or branched hydrocarbon groups. Suitable alkyl groups include, but are not limited to methyl, ethyl, propyl, isopropyl, butyl, isobutyl, pentyl, hexyl, heptyl, octyl, nonyl, decyl, dodecyl, tetradecyl, hexadecyl and octadecyl. The term alkyl may be prefixed by a specified number of carbon atoms to indicate the number of carbon atoms or a range of numbers of carbon atoms that may be present in the alkyl group. For example, C₁-C₃alkyl refers to methyl, ethyl, propyl and isopropyl.

The term “cycloalkyl” as used herein, refers to cyclic hydrocarbon groups. Suitable cycloalkyl groups include, but are not limited to, cyclopropyl, cyclobutyl, cyclopentyl, cyclohexyl, cycloheptyl, cyclooctyl, cyclononyl, cyclodecyl, cycloundecyl and cyclododecyl.

The term “heterocyclyl” as used herein refers to 5 or 6 membered saturated, partially unsaturated or aromatic cyclic hydrocarbon groups in which at least one carbon atom has been replaced by N, O or S. Optionally, the heterocyclyl group may be fused to a phenyl ring. Suitable heterocyclyl groups include, but are not limited to pyrrolidinyl, piperidinyl, pyrrolyl, thiophenyl, furanyl, oxazolyl, imidazolyl, thiazolyl, isoxazolyl, pyrazolyl, isothiazolyl, pyridinyl, quinolinyl, isoquinolinyl, indolyl, benzofuranyl, benzothiophenyl, oxadiazolyl, tetrazolyl, triazolyl and pyrimidinyl.

The term “aryl” as used herein, refers to C₆-C₁₀ aromatic hydrocarbon groups, for example phenyl and naphthyl.

As used herein, the term “flocculation” refers to the process in which two droplets in an emulsion come into contact with one another and become adhered to each other.

As used herein, the term “coalescence” refers to the process in which two droplets in an emulsion come into contact with one another and merge to form a single droplet.

As used herein, the term “phase separation” refers to the occurrence of multiple coalescence events resulting in the two phases of the emulsion separating.

As used herein, the term “salt” refers to a soluble chemical entity in which positive and negative ions combine to give a substance without a net positive or negative charge.

The terms “ion” and “ionic” refer to a chemical species bearing a positive or negative charge. Ions bearing a positive charge are referred to as cations or being cationic. Ions bearing a negative charge are referred to as anions or being anionic. Ions bearing both a positive and negative charge are referred to as zwitterions or ampholytes or being zwitterionic. The ions may be a single atom or a group of atoms and may be monovalent, for example, Na⁺ or NH₄⁺, or multivalent, for example, Ca⁺⁺ or PO₄³⁻. Upon solubilization, the ions of a salt may dissociate.

As used herein, the term “hydration energy” refers to the amount of energy released on addition of one mole of a solute to water to give an infinitely dilute solution. A large hydration energy corresponds to strong interactions between the solute and water. The hydration energy is generally reported as the Gibbs free energy of hydration (ΔG_{hyd}), which is strongly negative for favourable interactions between the solute and water. Other physical parameters of interest include the molar enthalpy of hydration (ΔH_{hyd}), which is also strongly negative for favourable interactions between the solute and water, and the molar entropy of hydration (ΔS_{hyd}), which is positive when ion hydration leads to a loss of order. The Gibbs free energy and enthalpy of hydration may be reported in kcal mol⁻¹ or kJ mol⁻¹. The entropy of hydration may be reported in cal mol⁻¹ K⁻¹ or J mol⁻¹ K⁻¹. The relationship between the Gibbs free energy of hydration, molar enthalpy of hydration and molar entropy of hydration is given by:

ΔG_{hyd}=ΔH_{hyd}−T·ΔS_{hyd} (Equation 1),

where T is the temperature in degrees Kelvin. Values for some selected ions are given in Table 3. It may be readily seen that strongly negative Gibbs free energies of hydration occur for small and/or multiply charged ions, and that the largest contribution to the Gibbs free energy of hydration derives from the enthalpy of hydration. A large negative Gibbs free energy of hydration also correlates with a more negative molar entropy of hydration, for example because of increased ordering of water molecules involved in ion hydration. Strongly-hydrated ions, such as carboxylate, carbonate or barium, show a tendency to form insoluble salts, particularly in the presence of strongly-hydrated counterions. This property has limited the utility of natural soaps (i.e. long-chain fatty acids containing carboxylate groups) as emulsifiers, because of the tendency of the strongly-hydrated carboxylate groups to form insoluble complexes with strongly hydrated cations such as magnesium, calcium or sodium (Zhang, Dery et al. 2004; Lin, McCormick et al. 2005). This loss of solubility leads to a loss of emulsifying activity in hard water or sea water, which have high concentrations of these ions. The present inventor has surprisingly found that peptide emulsifiers containing carboxylate groups retain their solubility and emulsifying activity in the presence of strongly-hydrated ions, unlike natural soaps.

TABLE 3
Gibbs free energies, molar enthalpies and molar entropies of hydration
of selected ions (taken from Marcus 1986; 1987; 1991)

| | ΔG_{hyd} | ΔH_{hyd} | ΔS_{hyd} |
|---|---|---|---|
| Ion | (kJ mol⁻¹) | (kJ mol⁻¹) | (J mol⁻¹ K⁻¹) |
| Mg²⁺ | −1830 | −1949 | n.a. |
| Ca²⁺ | −1505 | −1602 | n.a. |
| Ba²⁺ | −1250 | −1332 | −205 |
| Li⁺ | −475 | −531 | −142 |
| Na⁺ | −365 | −416 | −111 |
| K⁺ | −295 | −334 | −74 |
| NH₄⁺ | −285 | −329 | −112 |
| PO₄³⁻ | −2765 | −2879 | n.a. |
| CO₃²⁻ | −1315 | −1397 | n.a. |
| SO₄²⁻ | −1080 | −1035 | −200 |
| F⁻ | −465 | −510 | −137 |
| Cl⁻ | −340 | −367 | −75 |
| Br⁻ | −315 | −336 | −59 |
| NO₃⁻ | −300 | −312 | −76 |
| I⁻ | −275 | −291 | −36 |
| SCN⁻ | −280 | −311 | −66 |
| ClO₄⁻ | −430 | −246 | −57 |
| BF₄⁻ | −190 | −227 | −66 |

As used herein, the term “Jones-Dole viscosity B coefficient” refers to a parameter relating the concentration of a salt in water to the viscosity of the salt solution. The viscosity of a salt solution can be readily measured, for example by determining the time required for the solution to flow through a small hole in the bottom of a tube. The results can be fitted to the following polynomial in c, the concentration of the salt, up to about 0.1 M for strong electrolytes with a 1:1 ion ratio (e.g. NaCl):

$$\frac{η}{{η}_{0}} = 1 + A \sqrt{c} + B \cdot c , \qquad ( Equation 2 )$$

where η is the viscosity of the salt solution, η₀ is the viscosity of pure water at the same temperature, A is an electrostatic term that is close to 1 for moderate salt concentrations, and B is the Jones-Dole viscosity B coefficient, a direct measure of the strength of ion-water interactions, normalized to the strength of water-water interactions in bulk solution (Collins 2004). Some representative values of Jones-Dole viscosity B coefficients are given in Table 4, whereby it may be seen that acetate ion containing a carboxylate group has a strongly positive Jones-Dole viscosity B coefficient, reflecting strong hydration of this singly-charged ion.

TABLE 4
Jones-Dole viscosity B coefficients of selected ions (Collins 2004)

| | B |
|---|---|
| Cation | |
| Mg²⁺ | 0.385 |
| Ca²⁺ | 0.285 |
| Ba²⁺ | 0.22 |
| Li⁺ | 0.150 |
| Na⁺ | 0.086 |
| K⁺ | −0.007 |
| NH₄⁺ | −0.007 |
| Rb⁺ | −0.030 |
| Cs⁺ | −0.045 |
| Anion | |
| PO₄³⁻ | 0.590 |
| CH₃COO⁻ | 0.250 |
| SO₄²⁻ | 0.208 |
| F⁻ | 0.100 |
| Cl⁻ | −0.007 |
| Br⁻ | −0.032 |
| NO₃⁻ | −0.046 |
| ClO₄⁻ | −0.061 |
| I⁻ | −0.068 |
| SCN⁻ | −0.103 |

As used herein, the term “zeta potential” refers to a measure of the magnitude of the electrostatic repulsion or attraction between colloidal particles. It is derived from electrophoretic mobility measurements and represents the electric potential in the interfacial double layer at the slipping plane relative the bulk fluid. A value of 25 mV (positive or negative) can be taken as the arbitrary value that separates low-charged surfaces from highly-charged surfaces. For particles that are small enough, a high zeta potential will confer stability, i.e. a solution or dispersion will resist aggregation. Colloids with a high zeta potential (negative or positive) are electrically stabilized while colloids with a low zeta potential tend to flocculate.

As used herein, the term “micelle” refers to a surfactant aggregate containing commonly 70-80 monomers, in which the surfactant headgroups are exposed to the polar solvent, while the hydrophobic tails aggregate to form an unstructured core. Small micelles are roughly spherical, but under high salt concentrations or in the presence of additives, “micelle growth” may occur leading to elongated wormlike micelles that may be less soluble in water than either spherical micelles or non-aggregated surfactant molecules.

The term “HLB” refers to the hydrophobic-lipophilic balance of a surfactant. This term is a measure of the degree of hydrophilicity or lipophilicity of a surfactant. The HLB value is obtained by dividing the mass of the hydrophilic portion of the surfactant molecule with the mass of the whole surfactant molecule. An HLB of 0 indicates a completely hydrophobic molecule while an HLB of 20 indicates a completely hydrophilic molecule. Typically an HLB value of 4-6 indicates the surfactant may act as a water-in-oil emulsifier and an HLB value of 8-18 indicates the surfactant molecule may act as an oil-in-water emulsifier.

### Methods of Conferring Salt-Resistance

In one aspect the present invention provides a method of stabilizing an oil-in-water emulsion against flocculation or coalescence, said method comprising forming the emulsion in the presence of at least one peptide emulsifier, said at least one peptide emulsifier comprising one or more amino acid residues with a side chain carboxylate group.

In some embodiments, the emulsion is an oil-in-water emulsion or a water-in-oil emulsion, especially an oil-in-water emulsion.

In the present invention, the peptide emulsifier imparts resistance to flocculation or coalescence in an emulsion to the destabilizing effects of salt.

In some embodiments, the peptide used as the emulsifier includes 2 to about 80 amino acid residues, especially 5 to about 60 amino acid residues and more especially 5 to 40 or 10 to about 40 amino acid residues in length.

The peptide emulsifiers of the invention include one or more amino acid residues with a side chain carboxylate group, especially more than one amino acid residues with a side chain carboxylate group, more especially 2 to 10, for example, 2 to 8, especially 2 to 6 or 4 to 8 amino acid residues with a side chain carboxylate group.

The peptide emulsifiers have an amphipathic character including amino acid residues having side chains with hydrophilic character and amino acid residues having side chains with hydrophobic character. The amino acid residues are arranged in sequence to provide amphipathic character and to give rise to an affinity for the liquid-liquid interface. In some embodiments, the peptides have ordered secondary structure such as α-helical structure or β-strand structure.

A β-strand peptide typically includes 5-10 amino acid residues and can bind at a liquid-liquid interface in a backbone conformation that is almost fully extended. In the present invention, β-strand peptides have alternating hydrophilic and hydrophobic amino acid residues.

An α-helical peptide is typically at least 5 amino acid residues in length. An α-helix has a perioidicity of 3.6 amino acid residues per turn of the helix. Thus, for an α-helical secondary structure, amphipathic character may be generated by including an amino acid residue with a hydrophobic side chain every three to four residues to provide an α-helix with a hydrophobic face. A Table (taken from Jones and Middelberg, 2002) showing a scale of hydrophobicity for twenty naturally occurring amino acid residues is given below (Table 5).

TABLE 5

| | | | secondary structure formation | |
|---|---|---|---|---|
| amino acid | charge | hydrophobicity | α-helix | β-sheet |
| alanine | | 0.616 | 1.45 | 0.97 |
| cysteine | | 0.680 | 0.77 | 1.30 |
| aspartate | − | 0.028 | 0.98 | 0.80 |
| glutamate | − | 0.043 | 1.53 | 0.26 |
| phenylalanine | | 1.000 | 1.12 | 1.28 |
| glycine | | 0.501 | 0.53 | 0.81 |
| histidine | + | 0.165 | 1.24 | 0.71 |
| isoleucine | | 0.943 | 1.00 | 1.60 |
| lysine | + | 0.283 | 1.07 | 0.75 |
| leucine | | 0.943 | 1.34 | 1.22 |
| methionine | | 0.738 | 1.20 | 1.67 |
| asparagine | | 0.236 | 0.73 | 0.65 |
| proline | | 0.711 | 0.59 | 0.62 |
| glutamine | | 0.251 | 1.17 | 1.23 |
| arginine | + | 0.000 | 0.79 | 0.90 |
| serine | | 0.359 | 0.79 | 0.72 |
| threonine | | 0.450 | 0.82 | 1.20 |
| valine | | 0.825 | 1.14 | 1.65 |
| tryptophan | | 0.878 | 1.14 | 1.19 |
| tyrosine | | 0.880 | 0.61 | 1.29 |

In some embodiments, the peptide emulsifiers comprise a suitable pattern of hydrophobic and hydrophilic amino acid residues to form an α-helical peptide and have a repeating sequence unit (abcdefg)ₙ wherein n is an integer from 2 to 12, especially 2 to 6, more especially 2 to 4. In the repeating unit (abcdefg), referred to as a heptad, residues a and d are hydrophobic residues. These hydrophobic residues appear on one face of the α-helix and are able to interact with hydrophobic moieties such as the oil phase of the emulsion. In the heptad, it is not necessary that the repeating unit begins at residue “a” provided that the sequence a to g is maintained and individual residues are not substituted with one another. For example, the heptad may be (abcdefg), (gabcdef), (cdefgab) and the like.

In the heptad described above, at least one of, especially both of, residues e and g are residues having carboxylate group in their side chain, such as glutamate and aspartate.

Residues b, c and f of the heptad may be any amino acid residue and may be selected for polar or non-polar or charged properties.

An example of a peptide emulsifier containing a repeating heptad (gabcdef)₃ is SEQ ID NO:3 (EISALEA)₃. In this peptide e and g are glutamate, a is isoleucine and d is leucine.

In some embodiments, the peptide emulsifiers comprise an α-helical peptide sequence which is an 18-mer as shown in FIG. 1. The peptide emulsifier may include one 18-mer unit and optionally further C-terminal and/or N-terminal amino acid residues, or the 18-mer may be a repeating unit where it is repeated two or three times. As for the heptad sequence described above, the 18-mer sequence does not have to begin at 1, it may begin at any point in the sequence provided the type of amino acid at each position is maintained. For example, the 18-mer may be 1 2 3 4 5 6 7 8 9 10 11 12 13 14 15 16 17 18, 5 6 7 8 9 10 11 12 13 14 15 16 17 18 1 2 3 4 or 10 11 12 13 14 15 16 17 18 1 2 3 4 5 6 7 8 9 or the like. An 18-mer scaffold having an α-helical structure advantageously provides good control of the positioning of the amino acid side chains.

FIG. 1 represents an end view along the length of a linear α-helical peptide showing the theoretical positions of the side chains of each residue designated 1 (closest to the end from which the view is taken) to 18 (the residue at the far end of the peptide). It can be seen that residues 1, 4, 8, 11, 15 and 18 are preferred sites for hydrophobic side chains and residues 7 and 12 may also have hydrophobic side chains. Residues 2, 6, 9, 10, 13 and 17 are preferred sites for hydrophilic residues and residues 3 and 16 may also have hydrophilic side chains. Residues 5 and 14 may have hydrophilic or hydrophobic side chains.

In some embodiments, at least one of the one or more carboxylate groups on the peptides is located in a lateral position with respect to the liquid-liquid interface, such as positions 3, 5, 7, 12, 14 and 16 of FIG. 1, rather than being directed away from the interface and into the bulk aqueous phase. In some embodiments, between 2 and 6 or 4 and 8 carboxylate groups are located in a lateral position with respect to the liquid-liquid interface. In some embodiments, one to six carboxylate groups are located in a lateral position with respect to the liquid-liquid interface and optionally one or more further carboxylate groups are directed away from the liquid-liquid interface, into the bulk aqueous phase, for example at positions 2, 6, 9, 10, 13 or 17.

Some exemplary α-helical peptide emulsifiers of the invention include:

SEQ ID NO: 1
Ac-MEELADS LEELARQ VEELESA-CONH₂

SEQ ID NO:1 conforms with the peptide of FIG. 1 where residues 2, 3, 6, 9, 10, 16 and 17 have side chains containing carboxylate groups (E or D) and residues 1, 4, 8, 11, 15 and 18 are hydrophobic residues.

SEQ ID NO: 2
Ac-LEELADS LEELAEQ VEELLSA-CONH₂

SEQ ID NO:2 conforms with a peptide of FIG. 1 where residues 2, 3, 6, 9, 10, 13, 16 and 17 have side chains containing carboxylate groups (E or D) and residues 1, 4, 8, 11, 15 and 18 are hydrophobic residues.

SEQ ID NO: 3
Ac-EISALEA EISALEA EISALEA-CONH₂

SEQ ID NO:3 is a repeating heptad unit (gabcdef)₃ in which a and d are hydrophobic and e and g are residues comprising a carboxylate containing side chain.

SEQ ID NO: 4
Ac-AISELEA EISALEA EIESLAA-CONH₂

SEQ ID NO:4 conforms with a peptide of FIG. 1 where residues 3, 5, 7, 12, 14 and 16 have side chains containing carboxylate groups (E) and residues 1, 4, 8, 11, 15 and 18 are hydrophobic residues. Residue 1 is considered to be the first isoleucine residue (I).

SEQ ID NO: 5
Ac-AIESLAE SIEELAE AISELAA-CONH₂

SEQ ID NO:5 conforms with a peptide of FIG. 1 where residues 2, 6, 9, 10, 13 and 17 have side chains containing carboxylate groups (E) and residues 1, 4, 8, 11, 15 and 18 are hydrophobic residues. Residue 1 is considered to be the first isoleucine residue (I).

SEQ ID NO: 6
H₂N-P LAEIDSA LAEIEAQ VAELIAA VED-CO₂H

SEQ ID NO:6 conforms with a peptide of FIG. 1 where residues 3, 5, 10, 12 and 17 have side chains containing carboxylate groups (E or D) and residues 1, 4, 8, 11, 15 and 18 are hydrophobic residues. Residue 1 is considered to be the first leucine residue (L).

SEQ ID NO: 7
H₂N-P LEAIADS LEAIAEQ VEALIEA VAD-CO₂H

SEQ ID NO:7 conforms with a peptide of FIG. 1 where residues 2, 6, 9, 13 and 16 have side chains containing carboxylate groups (E or D) and residues 1, 4, 8, 11, 15 and 18 are hydrophobic residues. Residue 1 is considered to be the first leucine residue (L).

SEQ ID NO: 8
H₂N-PG IAELEAE LSAVEAE LEAILAE LD-CO₂H

SEQ ID NO:8 conforms with a peptide of FIG. 1 where residues 3, 5, 7, 12, 14 and 16 have side chains containing carboxylate groups (E) and residues 1, 4, 8, 11, 15 and 18 are hydrophobic residues. Residue 1 is considered to be the first isoleucine residue (I).

SEQ ID NO: 9
Ac-LAELESL LAELEAL VAELLSA-CONH₂

SEQ ID NO:9 conforms with a peptide of FIG. 1 where residues 3, 5, 10, 12 and 17 have side chains containing carboxylate groups (E) and residues 1, 4, 8, 11, 15 and 18 are hydrophobic residues.

SEQ ID NOs:10 and 11 are β-strand peptides having predominantly alternating hydrophobic and hydrophilic residues.

SEQ ID NO: 10
Ac-PDFDFDFDP-CONH₂
SEQ ID NO: 11
Ac-PEFEFEFEP-CONH₂

The at least one peptide emulsifier may be in, the form of a mixture of emulsifiers. The mixture may contain more than one peptide emulsifier, each comprising one or more amino acid residues with a side chain carboxylate group.

The mixture may contain one or more peptide emulsifiers comprising one or more amino acid residues with a side chain carboxylate group, together with other amphipathic peptides that have an affinity for the liquid-liquid interface. In this case, the peptide emulsifiers present in the mixture but do not contain one or more amino acid residues with a side chain carboxylate group preferably lack amino acid residues with strongly chaotropic or weakly hydrated charged side chains such as arginine or histidine. In some embodiments, the peptide emulsifiers that do not include one or more amino acid residues with a side chain carboxylate acid, include lysine amino acid residues having a cationic side chain and/or uncharged polar amino acid residues such as serine, threonine, asparagine and glutamine as the polar amino acids in their amphipathic structure.

Within a mixture of peptide emulsifiers, at least 10% of the peptides must have one or more amino acid residues with a side chain carboxylate group, especially at least 30%, more especially at least 50%.

Within a mixture of peptide emulsifiers, different peptides may have different affinities for the liquid-liquid interface therefore the population of peptides at the interface may not reflect the population of peptides in the bulk aqueous phase. For example, if the mixture of peptide emulsifiers in the aqueous phase is 50% peptide A and 50% peptide B, the population at the liquid-liquid interface could be 90% peptide A and 10% peptide B as peptide A has a higher affinity for the liquid-liquid interface.

In some embodiments, other non-peptidic emulsifiers may be included in the mixture, provided these emulsifiers do not contain weakly hydrated groups such as sulfate, sulfonate, alkylamine or guanidinium ions. Suitable non-peptide emulsifiers that may be present include PEGylated surfactants, surfactants based on sugar groups such as octyl glucoside, and surfactants containing carboxylate groups.

The presence of salt in an aqueous phase of an emulsion or in the bulk aqueous phase before an emulsion is formed can destabilize the emulsion or prevent the emulsion being formed, particularly if high salt concentrations are present.

The stability of an emulsion to flocculation or coalescence in the presence of a salt depends on whether an emulsifier is present, the identity of the emulsifier, the density of the oil and water phases, the dispersed phase volume fraction, the size of the initial emulsion droplets, the identity of the salt and the amount of salt present. In general, emulsions formed in the absence of an emulsifier are unstable and may flocculate or coalesce in the presence of small amounts of salt, or the emulsion may not form at all if the salt is present in the aqueous phase at the time of emulsion formation. Emulsifiers may be used to stabilize an emulsion to flocculation or coalescence. However, some emulsifiers are unable to maintain stability of an emulsion in the presence of a salt or the emulsifier stabilized emulsion may become increasingly unstable with increasing amounts of salt. In some cases an emulsifier may precipitate in the presence of a salt, for example, a fatty acid surfactant has a high tendency to precipitate in the presence of salts, particularly salts containing Ca⁺⁺ or Mg⁺⁺ ions as are found in hard water.

The peptide emulsifiers provide resistance to flocculation or coalescence in an emulsion in the presence of a salt compared to emulsions with no emulsifiers or emulsions formed only with other types of emulsifiers such as sulfate, sulfonate, fatty acid-containing emulsifiers, cationic emulsifiers such as primary, secondary, tertiary or quaternary amino-containing emulsifiers or certain non-ionic emulsifiers, such as emulsifiers containing poly(ethyleneglycol) polar groups. In general, the peptide emulsifiers of the present invention improve stability of an emulsion to flocculation or coalescence or improve resistance to flocculation or coalescence in the presence of salts that have positive and negative ions that are each a single atom or a small group of atoms such as 2 to 10 atoms, especially 2 to 6 atoms, or mixtures thereof. In some embodiments, the ions in the salt are inorganic ions.

In some embodiments, the salt comprises ions selected from Mg²⁺, Ca²⁺, Ba²⁺, Li⁺, Na⁺, K⁺, NH₄⁺, PO₄³⁻, CO₃²⁻, SO₄²⁻, Cl⁻, Br⁻, I⁻, SCN⁻, ClO₄⁻ and BF₄⁻, especially Na⁺, K⁺, Ca²⁺, Mg²⁺, PO₄³⁻, CO₃²⁻, SO₄²⁻, Cl⁻ and NO₃⁻.

The term “stabilizing an emulsion against flocculation or coalescence” does not necessarily mean that the emulsion is permanently stabilized and will never flocculate or coalesce. The stabilizing may be a temporary stabilizing of the emulsion where flocculation or coalescence may readily occur if the conditions in the emulsion are altered. Alternatively, the stabilization may be an improved stabilization compared to the total absence of an emulsifier or the use of a emulsifier other than an emulsifier of the present invention. For example, the extent of flocculation or coalescence is reduced, the onset of flocculation or coalescence may be delayed or a stable emulsion may be formed in the presence of an emulsifier of the present invention. An improvement may also be no or less flocculation or coalescence over a given period of time compared to the absence of an emulsifier or the presence of an emulsifier other than an emulsifier of the present invention. In another example, the emulsion comprising the peptide emulsifier of the present invention has improved resistance to flocculation or coalescence in the presence of salt, particularly amounts of salt that would cause flocculation or coalescence in the absence of an emulsifier other than the peptide emulsifier of the present invention. In some cases, the peptide emulsifiers impart resistance to flocculation and coalescence on an emulsion in the presence of higher concentrations of salt than would normally cause flocculation or coalescence in the absence of an emulsifier or with only an emulsifier other than an emulsifier of the present invention, such as a sulfate, sulfonate, fatty acid-containing emulsifier, cationic emulsifiers such as primary, secondary, tertiary or quaternary amino-containing emulsifiers or certain non-ionic emulsifiers, such as emulsifiers containing poly(ethyleneglycol) polar groups.

The minimum size of surfactant-saturated emulsion droplets that can be produced in an oil-in-water emulsion for a given oil volume fraction and emulsifier concentration is given by:

$${d}_{min} = \frac{6 \cdot {Γ}_{sat} \cdot ϕ}{{C}_{s}} \qquad ( Equation 3 )$$

where dₘᵢₙ is the minimum droplet diameter (in m), Γₛₐₜ is the excess surface concentration of the emulsifier (in kg m⁻²), φ is the oil volume fraction and Cₛ is the emulsifier concentration in the total emulsion (in kg m⁻³). The relation may also be given as:

$${d}_{min} = \frac{6 \cdot {Γ}_{sat} \cdot φ}{{C}_{s}^{′} \cdot ( 1 - ϕ )} \qquad ( Equation 4 )$$

where C′ₛ is the surfactant concentration in the aqueous phase. The relations are given for the case of a monodisperse emulsion, in which all droplets have the same size. For a polydisperse emulsion in which emulsion droplets have different sizes, the appropriate diameter is the volume-surface mean diameter or Sauter mean diameter:

$${d}_{32} = \frac{{\sum }_{i = 1}^{ } {n}_{i} {d}_{i}^{3}}{{\sum }_{i = 1}^{ } {n}_{i} {d}_{i}^{2}} \qquad ( Equation 5 )$$

which can be calculated from a table of size distribution frequencies, as available from commercial sizing instruments such as the Nano Zetasizer ZS (Malvern Instruments). Oils suitable for use in the invention are any oils that have poor solubility in water. Suitable oils include, but are not limited to, hydrocarbons including hexane, heptane, octane, decane, dodecane, tetradecane, hexadecane, octadecane, benzene and toluene; halogenated hydrocarbons including dichloromethane, chloroform and carbon tetrachoride; mineral oils including paraffinic, naphthenic or aromatic oils; crude oils; silicone oils; synthetic oils including poly-alphaolefins and synthetic esters; vegetable oils including olive oil, peanut oil, sesame oil, sunflower oil, cottonseed oil, caster oil, rapeseed oil, palm oil, soybean oil, coconut oil, and blended oils; synthetic triglycerides; alpha-tocopherol and terpenes. Oils suitable for use in the invention may also include fluorinated or perfluorinated analogs of oils listed herein.

Aqueous phases that are suitable for use in the invention are any polar solvent in which the peptide emulsifier is soluble. For example, suitable aqueous phases include water or mixtures of water with other polar miscible solvents such as methanol and ethanol. The aqueous phase may also be a buffer solution such as borate buffer, Tris buffer, citrate buffer, acetate buffer, formate buffer, phosphate buffer or HEPES buffer.

In some embodiments, the aqueous phase of the emulsion is derived from sea water, brine, saline or hard water which all include high or moderate amounts of salt.

The aqueous phase may be at any pH at which the peptide emulsifier is soluble. Generally the pH of the aqueous phase will be at least 5, especially at least 6, more especially at least 7. In some instances the pH of the aqueous phase is about 8. Peptide emulsifier dissolution occurs when a sufficient number of carboxyl groups have dissociated to carboxylate groups with a negative charge and hydrophobic interactions between peptide molecules in the solid are overcome. Dissociation of carboxyl groups occurs over a range of pH values due to ionic interaction of different carboxylate groups with one another, and may occur well above the monomer pKa of about 4.5 (for glutamic acid).

In another aspect of the invention there is provided a method of making an emulsion resistant to flocculation or coalescence comprising:
mixing an aqueous phase and an oil phase together in the presence of at least one peptide emulsifier comprising one or more amino acid residues with a side chain carboxylate group.

The emulsions of the invention may be prepared by any known means of forming emulsions by mixing the aqueous and oil phases together. The mixing may be achieved by agitation of the two liquids by stirring or shaking, by homogenization, applying shear or by pumping the two liquids into a container at high speed or pressure. The vigorousness of mixing will determine the speed at which the emulsion forms and the size of the droplets in the dispersed liquid phase as is known in the art of emulsion formation.

In some embodiments, the at least one peptide emulsifier is solubilised in the aqueous phase before mixing.

In some embodiments, the peptide emulsifier is in a mixture of peptide emulsifiers, at least one of which comprises one or more amino acid residues with a side chain carboxylate group.

In yet another aspect, the present invention provides a salt-resistant emulsion comprising at least one peptide emulsifier comprising one or more amino acid residues with a side chain carboxylate group.

In some embodiments, the peptide emulsifier is an α-helical peptide. In other embodiments, the peptide emulsifier is a β-strand peptide. In some embodiments, the one or more carboxylate groups of the peptide emulsifier are oriented around the liquid-liquid interface of the emulsion. In some embodiments, the peptide emulsifier is selected from a peptide of SEQ ID NOs: 1 to 11.

In some embodiments, the peptide emulsifier is in a mixture of peptide emulsifiers, at least one of which comprises one or more amino acid residues with a side chain carboxylate group.

In yet a further aspect, the present invention provides a peptide having one of the following sequences:

SEQ ID NO: 2
Ac-LEELADS LEELAEQ VEELLSA-CONH₂
SEQ ID NO: 3
Ac-EISALEA EISALEA EISALEA-CONH₂
SEQ ID NO: 4
Ac-AISELEA EISALEA EIESLAA-CONH₂
SEQ ID NO: 5
Ac-AIESLAE SIEELAE AISELAA-CONH₂
SEQ ID NO: 6
H₂N-P LAEIADS LAEIAEQ VAELIEA VED-CO₂H
SEQ ID NO: 7
H₂N-P LEAIADS LEAIAEQ VEALIEA VAD-CO₂H
SEQ ID NO: 8
H₂N-PG IAELEAE LSAVAEA LEAILAE LD-CO₂H
SEQ ID NO: 9
Ac-LAELESL LAELEAL VAELLSA-CONH₂.

### Applications

Emulsion stability in the presence of salts may be useful in applications such as beverages, processed foods, pharmaceuticals, cosmetics, inks and printing, paints, and coatings, surfactants, waste water treatment, explosives, bioremediation, corrosion inhibition, drilling, oil recovery, medicine, dentistry, biocatalysis and biotechnology. In general, any emulsion-based application in which the presence of salts in general, or calcium or magnesium salts in particular, results in an undesired loss of emulsion stability, may be a suitable application for the use of the peptide emulsifiers described in this invention.

The invention may be useful in formulating emulsions for intravenous (i.v.) delivery of active pharmaceutical agents, in which an emulsion will come into contact with plasma containing high concentrations of sodium and chloride and moderate concentrations of potassium, calcium, magnesium and bicarbonate. Contact with these salts in plasma will cause flocculation and/or coalescence of many i.v. emulsion formulations, potentially blocking blood vessels and causing life-threatening complications. For this reason, as well as for reasons of intrinsic surfactant toxicity, it is frequently difficult to identify conventional emulsifiers that are suitable for the formulation of i.v. emulsions. Peptide emulsifiers as described in this invention may offer one solution to this problem.

The invention may also be useful in formulating explosive emulsions. In some cases, an explosive is prepared by emulsifying an aqueous phase containing high concentrations of an oxidizing salt, such as sodium nitrate, with an oil phase as the explosive fuel. In some cases, use of a salt-resistant emulsifier such as described in this invention may be useful in preparing alternate formulations of explosive emulsions.

The invention may also be useful in formulating emulsions for use in the oil industry, such as drilling fluids. In some cases, drilling through geological formations to reach oil reservoirs may involve contact with brine or solid salt formations that can destabilize emulsions formulated with conventional surfactants, causing failure of the drilling fluid. The use of salt-resistant emulsifiers such as those described in the current invention may assist in the formulation of salt-resistant drilling fluids.

The invention may further be useful in preparing stimulation fluids for use in the oil industry during enhanced oil recovery operations. In some cases, flushing of an oil reservoir may be carried out using an aqueous solution of surfactant that may come into contact with brine or solid salt formations that can destabilize emulsions formed between a conventional stimulation fluid and oil in the reservoir, resulting in a diminished flushing of oil from the reservoir. The use of salt-resistant emulsifiers such as those described in the current invention may assist in the flushing of oil from oil reservoirs during enhanced oil recovery operations.

The invention may also be useful in the preparation of food and beverage emulsions that are stable in the presence of high concentrations of salt, added as a flavouring agent.

The invention may also be useful in the stabilization of emulsions, such as cutting fluid emulsions, that may experience high salt concentrations as a result of evaporation of water from the initial formulation and/or dilution with local water containing moderate concentrations of calcium or magnesium salts (hard water).

### BRIEF DESCRIPTION OF THE FIGURES

FIG. 1 is a schematic representation of an α-helical peptide having 18 amino acid residues with an end view along the length of the peptide and showing the positions of the amino acid residue side chains.

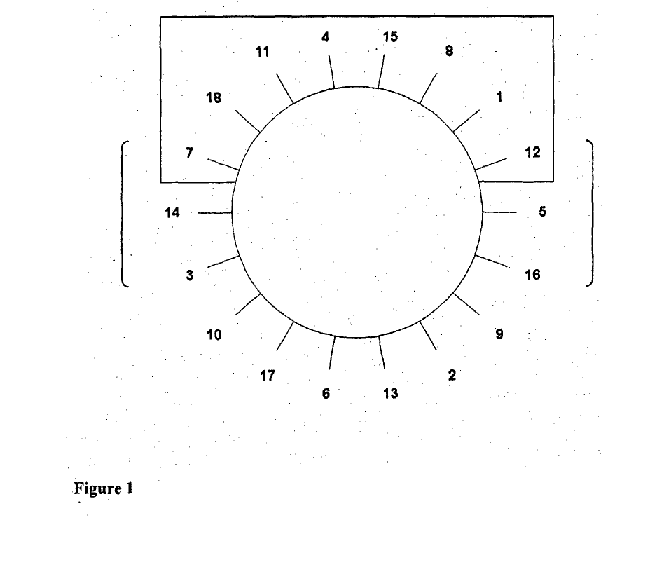

### EXAMPLES

### Reagents

Reagents were analytical grade unless otherwise indicated. Ultrapure water for cleaning and solution preparation was produced using a MilliQ water purification unit (Millipore, North Ryde, NSW, Australia) and had a resistivity of >18.2 MΩcm. Glassware was cleaned by soaking in 2% (v/v) Decon90 (Decon Laboratories Ltd, Hove, East Sussex, UK), rinsed extensively with water, soaked for 10 min in freshly prepared piranha solution (equal parts of 30% (v/v) H₂O₂ and 98% (v/v) H₂SO₄), then rinsed with copious amounts of water.

Hexadecane oil was purchased from Sigma-Aldrich and was cleaned of surface-active impurities by prolonged stirring with activated silica or aluminum oxide before use. Other oils included food-grade canola oil, methyl oleate (Agnique ME 181-U, Cognis Corporation, Cincinnati, Ohio, 76% methyl oleate, 24% miscellaneous methyl esters), Cropspray 11N (Petro-Lube, London, UK, heavy paraffinic oil used in formulating pesticides and herbicides), Solvesso 200ND (Exxon Mobil Chemical, Cologne, Germany, liquid aromatic hydrocarbons), Durasyn® 164 polyalpha-olefins (PAO4, Innovene, Middelsex, UK, 1-decene homopolymer, hydrogenated), Jurong 150 (Mobil Oil Australia, severely treated base oils) and Mobil 100 (AP/E core 100, Mobil Oil Australia, severely treated base oils). Peptides SEQ ID NOs:1-3 and SEQ ID NO:8 (95% purity) were custom synthesized by Genscript (Piscataway, N.J.). SEQ ID NOs:4-5 and SEQ ID NOs:10-11 (95% purity) and SEQ ID NOs:6-7 and SEQ ID NO:9 (desalted) were custom synthesized by Peptide 2.0 (Chantilly, Va.). Peptides were stored at −80° C. Dissolution of acidic peptides was achieved by addition of a minimum amount of inorganic (e.g. NaOH or KOH) or organic (e.g. triethanolamine) base to ionize the peptide carboxylate groups and adjust the pH to a desired final value.

### Emulsion Preparation

Oil-in-water emulsions were prepared by sonication of a peptide solution with a predetermined volume of oil using either the micro probe or 10 mm horn of a Branson Sonifier 250 or 450. Sonication was carried out for 1-4 cycles of 30 s each and the dispersion was allowed to rest for 10 minutes between sonication cycles. Droplet sizing and zeta potential measurements used a Zetasizer NanoZS (Malvern Instruments Ltd, Worcestershire, UK) following dilution of emulsion samples either in water or a salt solution, most commonly a salt solution equivalent to the solution in which the peptide was dissolved for emulsion preparation.

Expected minimum droplet sizes corresponding to given surface excesses were calculated via:

$${d}_{min} = \frac{6 \times {Γ}_{sat} \cdot Φ}{{C}_{s}} \qquad ( Equation 3 )$$

where dₘᵢₙ is the minimum droplet diameter (in m), Γₛₐₜ is the surface excess of surfactant (in mg m⁻²), Φ is the oil volume fraction in the emulsion (unitless) and Cₛ is the surfactant concentration in the total emulsion (mg m⁻³).

Where appropriate, solution ionic strengths (I) were determined using

$$I = \frac{1}{2} \cdot {\sum }_{i = 1}^{n} {c}_{i} \cdot {z}_{i}^{2} \qquad ( Equation 6 )$$

where c is the molar concentration of each ion and z is the ion charge.

### Example 1

### Preparation of Emulsions Using Peptide SEQ ID NO: 1. Effects of Sodium or Potassium Chloride

A series of emulsions was prepared by sonicating aqueous solutions of 1 mM SEQ ID NO: 1, 0.0-2.0 M NaCl or KCl, pH 6.0, with 20% (v/v) hexadecane. Samples of the emulsions were diluted in the corresponding salt solution and the droplet size distribution and zeta potential were determined using a Zetasizer Nano ZS. The Sauter mean diameter and zeta potential at selected salt concentrations are given in Table 6 Larger droplet sizes indicate flocculation and/or coalescence of droplets in the emulsion. It can be seen that sensitivity to high salt concentrations is greater with NaCl than KCl for this peptide emulsifier.

TABLE 6
Effect of salts on the Sauter mean diameter
of droplets in emulsions prepared with 1 mM
SEQ ID NO: 1 pH 6.0 and 20% (v/v) hexadecane.

| Salt Concentration | d₃₂ (nm) | Zeta potential (mV) |
|---|---|---|
| 0 mM (KCl) | 374 | −62.8 |
| 100 mM (KCl) | 314 | −37.2 |
| 200 mM (KCl) | 515 | −37.7 |
| 500 mM (KCl) | 569 | −24.4 |
| 1.0M (KCl) | 543 | −16.6 |
| 2.0M (KCl) | 291 | −13.4 |
| 100 mM (NaCl) | 282 | −40.3 |
| 200 mM (NaCl) | 289 | −39.7 |
| 500 mM (NaCl) | 454 | −16.3 |
| 1.0M (NaCl) | 4234 | −4.62 |
| 2.0M (NaCl) | 4471 | −12.4 |

### Example 2

### Preparation of Emulsions Using Peptide SEQ ID NO: 3. Effects of Sodium or Potassium Chloride

A series of emulsions was prepared by sonicating aqueous solutions of 1 mM SEQ ID NO: 3, 0.0-2.0 M NaCl or KCl, pH 9.0, with 20% (v/v) hexadecane. Samples of the emulsions were diluted in the corresponding salt compositions and the droplet size distribution was determined using a Zetasizer Nano ZS. The zeta potential was determined after dilution in water. The Sauter mean diameter and zeta potential at selected salt concentrations are given in Table 7. Larger droplet sizes indicate flocculation and/or coalescence of droplets in the emulsion. It can be seen that sensitivity to high salt concentrations is similar with NaCl and KCl for this peptide emulsifier.

TABLE 7
Effect of salts on the Sauter mean diameter and zeta
potential of droplets in emulsions prepared with 1
mM SEQ ID NO: 3 pH 9.0 and 20% (v/v) hexadecane.

| Salt Concentration | d₃₂ (nm) | Zeta potential (mV) |
|---|---|---|
| 0 mM (KCl) | 243 | −69.6 |
| 20 mM (KCl) | 236 | −65.5 |
| 50 mM (KCl) | 249 | −37.0 |
| 100 mM (KCl) | 280 | −27.7 |
| 200 mM (KCl) | 267 | −30.1 |
| 500 mM (KCl) | 253 | −23.4 |
| 1.0M (KCl) | 4028 | −25.4 |
| 2.0M (KCl) | 3035 | −17.1 |
| 200 mM (NaCl) | 240 | −32.6 |
| 2.0M (NaCl) | 4246 | −9.7 |

### Example 3

### Dilution of Emulsions Prepared Using Peptide SEQ ID NO: 7 in Salt Solutions. Effects of Sodium, Potassium or Calcium Chloride

An emulsion was prepared by sonicating an aqueous solution of 1 mM SEQ ID NO: 7 pH 6.0 with 20% (v/v) hexadecane. Samples of the emulsion were diluted in different salt compositions and the droplet size distribution was determined using a Zetasizer Nano ZS. The Sauter mean diameter at selected salt concentrations is given in Table 8. Larger droplet sizes indicate flocculation and/or coalescence of droplets in the emulsion. It can be seen, that an emulsion prepared at low salt concentrations with this peptide tolerates subsequent dilution in high concentrations of sodium, potassium or calcium chloride, but is more sensitive to sodium than to potassium chloride.

TABLE 8
Effect of dilution in different salt solutions on the Sauter
mean diameter of droplets in an emulsion prepared with
1 mM SEQ ID NO: 7 pH 6.0 and 20% (v/v) hexadecane.

| Diluent | d₃₂ (nm) |
|---|---|
| No added salt | 461 |
| 100 mM (KCl) | 424 |
| 200 mM (KCl) | 449 |
| 500 mM (KCl) | 486 |
| 1.0M (KCl) | 683 |
| 2.0M (KCl) | 598 |
| 100 mM (NaCl) | 406 |
| 200 mM (NaCl) | 417 |
| 500 mM (NaCl) | 466 |
| 1.0M (NaCl) | 538 |
| 2.0M (NaCl) | 3029 |
| 1 mM (CaCl₂) | 421 |
| 2 mM (CaCl₂) | 425 |
| 5 mM (CaCl₂) | 439 |
| 10 mM (CaCl₂) | 412 |
| 20 mM (CaCl₂) | 796 |
| 50 mM (CaCl₂) | 802 |
| 100 mM (CaCl₂) | 744 |

### Example 4

### Preparation of Emulsions Using Peptide SEQ ID NO: 6. Effects of Sodium or Potassium Chloride

A series of emulsions was prepared by sonicating aqueous solutions of 2 mM SEQ ID NO: 6, 0.0-2.0 M NaCl or KCl, pH 8.0, with 50% (v/v) hexadecane. Samples of the emulsions were diluted in the corresponding salt compositions and the droplet size distribution and zeta potential were determined using a Zetasizer Nano ZS. The Sauter mean diameter and zeta potential at selected salt concentrations are given in Table 9. Larger droplet sizes indicate flocculation and/or coalescence of droplets in the emulsion. It can be seen that sensitivity to high salt concentrations is similar with NaCl and KCl for this peptide emulsifier.

TABLE 9
Effect of salts on the Sauter mean diameter and zeta
potential of droplets in emulsions prepared with 2
mM SEQ ID NO: 6 pH 8.0 and 50% (v/v) hexadecane.

| Salt Concentration | d₃₂ (nm) | Zeta potential (mV) |
|---|---|---|
| 0 mM (KCl) | 972 | −61.3 |
| 100 mM (KCl) | 862 | −70.1 |
| 200 mM (KCl) | 846 | −53.9 |
| 500 mM (KCl) | 727 | −36.5 |
| 1.0M (KCl) | 874 | −27.4 |
| 2.0M (KCl) | 419 | −26.7 |
| 100 mM (NaCl) | 893 | −63.0 |
| 200 mM (NaCl) | 747 | −46.5 |
| 500 mM (NaCl) | 471 | −32.8 |
| 1.0M (NaCl) | 931 | −24.2 |
| 2.0M (NaCl) | 819 | −12.0 |

### Example 5

### Preparation of Emulsions Using Peptide SEQ ID NO: 7. Effects of Sodium or Potassium Chloride

A series of emulsions was prepared by sonicating aqueous solutions of 2 mM SEQ ID NO: 7, 0.0-2.0 M NaCl or KCl, pH 8.0, with 50% (v/v) hexadecane. Samples of the emulsions were diluted in the corresponding salt compositions and the droplet size distribution and zeta potential were determined using a Zetasizer Nano ZS. The Sauter mean diameter and zeta potential at selected salt concentrations are given in Table 10. Larger droplet sizes indicate flocculation and/or coalescence of droplets in the emulsion. It can be seen that sensitivity to high salt concentrations in general is greater for this peptide emulsifier than for SEQ ID NO: 6 and that sensitivity to NaCl is greater than that to KCl.

TABLE 10
Effect of salts on the Sauter mean diameter and zeta
potential of droplets in emulsions prepared with 2
mM SEQ ID NO: 7 pH 8.0 and 50% (v/v) hexadecane.

| Salt Concentration | d₃₂ (nm) | Zeta potential (mV) |
|---|---|---|
| 0 mM (KCl) | 932 | −67.2 |
| 100 mM (KCl) | 318 | −70.0 |
| 200 mM (KCl) | 907 | −59.6 |
| 500 mM (KCl) | 650 | −47.6 |
| 1.0M (KCl) | 516 | −36.2 |
| 2.0M (KCl) | 1611 | −23.8 |
| 100 mM (NaCl) | 651 | −64.2 |
| 200 mM (NaCl) | 703 | −50.2 |
| 500 mM (NaCl) | 694 | −33.2 |
| 1.0M (NaCl) | 2370 | −25.9 |
| 2.0M (NaCl) | 3443 | −13.5 |

### Example 6

### Preparation of Emulsions Using Peptide SEQ ID NO: 8. Effects of Ammonium Chloride or Sulfate

A series of emulsions was prepared by sonicating aqueous solutions of 1 mM SEQ ID NO: 8, 0.0-2.0 M NH₄Cl or (NH₄)₂SO₄, pH 8.0, with 20% (v/v) Cropspray 11N (industrial mineral oil). Samples of the emulsions were diluted in the corresponding salt compositions and the droplet size distribution and zeta potential were determined using a Zetasizer Nano ZS. The Sauter mean diameter and zeta potential at selected salt concentrations are given in Table 11. Larger droplet sizes indicate flocculation and/or coalescence of droplets in the emulsion. Sensitivity to high salt appears comparable between these two salts given that for equimolar salt concentrations, the ionic strength of an ammonium sulfate solution is three times higher than that of an ammonium chloride solution.

TABLE 11
Effect of salts on the Sauter mean diameter and zeta
potential of droplets in emulsions prepared with 1 mM
SEQ ID NO: 8 pH 8.0 and 20% (v/v) Cropspray 11N.

| Salt Concentration | d₃₂ (nm) | Zeta potential (mV) |
|---|---|---|
| 0 mM (NH₄Cl) | 1089 | −71.3 |
| 100 mM (NH₄Cl) | 1593 | −50.8 |
| 200 mM (NH₄Cl) | 947 | −36.3 |
| 500 mM (NH₄Cl) | 1084 | −22.0 |
| 1.0M (NH₄Cl) | 1074 | −15.4 |
| 2.0M (NH₄Cl) | 2889 | −13.1 |
| 100 mM ((NH₄)₂SO₄) | 838 | −39.9 |
| 200 mM ((NH₄)₂SO₄) | 735 | −24.8 |
| 500 mM ((NH₄)₂SO₄) | 1932 | −23.6 |
| 1.0M ((NH₄)₂SO₄) | 2797 | −20.4 |
| 2.0M ((NH₄)₂SO₄) | 3546 | −23.6 |

### Example 7

### Preparation of Emulsions Using Peptide SEQ ID NO: 4. Effects of Lithium Chloride or Sulfate

A series of emulsions was prepared by sonicating aqueous solutions of 1 mM SEQ ID NO: 4, 0.0-2.0 M LiCl or Li₂SO₄, pH 8.0, with 20% (v/v) PAO 4 (synthetic industrial oil). Samples of the emulsions were diluted in the corresponding salt compositions and the droplet size distribution was determined using a Zetasizer Nano ZS. The Sauter mean diameter at selected salt concentrations is given in Table 12. Larger droplet sizes indicate flocculation and/or coalescence of droplets in the emulsion. Sensitivity to high salt appears comparable between these two salts, although at some intermediate salt concentrations a smaller calculated Sauter mean diameter fails to reflect the presence of a population of larger droplets.

TABLE 12
Effect of salts on the Sauter mean diameter of droplets in emulsions
prepared with 1 mM SEQ ID NO: 4 pH 8.0 and 20% (v/v) PAO 4.

| Salt Concentration | d₃₂ (nm) |
|---|---|
| 0 mM (LiCl) | 2204 |
| 100 mM (LiCl) | 1191 |
| 200 mM (LiCl) | 878 |
| 500 mM (LiCl) | 2551 |
| 1.0M (LiCl) | 2257 |
| 2.0M (LiCl) | 2237 |
| 100 mM (Li₂SO₄) | 1816 |
| 200 mM (Li₂SO₄) | 3507 |
| 500 mM (Li₂SO₄) | 539 |
| 1.0M (Li₂SO₄) | 534 |
| 2.0M (Li₂SO₄) | 3052 |

### Example 8

### Preparation of Emulsions Using Peptide SEQ ID NO: 8. Effects of Sodium or Potassium Chloride

A series of emulsions was prepared by sonicating aqueous solutions of 1 mM SEQ ID NO: 8, 0.0-2.0 M NaCl or KCl, pH 8.0, with 20% (v/v) hexadecane. Samples of the emulsions were diluted in the corresponding salt compositions and the droplet size distribution and zeta potential were determined using a Zetasizer Nano ZS. The Sauter mean diameter and zeta potential at selected salt concentrations are given in Table 13. Larger droplet sizes indicate flocculation and/or coalescence of droplets in the emulsion. It can be seen that sensitivity to high salt concentrations is greater for NaCl than KCl for this peptide emulsifier.

TABLE 13
Effect of salts on the Sauter mean diameter and zeta
potential of droplets in emulsions prepared with 1
mM SEQ ID NO: 8 pH 8.0 and 20% (v/v) hexadecane.

| Salt Concentration | d₃₂ (nm) | Zeta potential (mV) |
|---|---|---|
| 0 mM (KCl) | 371 | −55.6 |
| 100 mM (KCl) | 287 | −43.1 |
| 200 mM (KCl) | 321 | −37.9 |
| 500 mM (KCl) | 382 | −27.4 |
| 1.0M (KCl) | 577 | −17.0 |
| 2.0M (KCl) | 3302 | −15.9 |
| 100 mM (NaCl) | 985 | −58.0 |
| 200 mM (NaCl) | 1277 | −40.9 |
| 500 mM (NaCl) | 2261 | −24.2 |
| 1.0M (NaCl) | 1042 | −13.0 |
| 2.0M (NaCl) | 3198 | −7.6 |

### Example 9

### Preparation of Emulsions of Crude Oil in Artificial Sea Water Using Peptides SEQ ID NOs: 4, 5 and 8

A series of emulsions was prepared by sonicating aqueous solutions of 2.5 mM SEQ ID NOs: 4, 5 or 8 in artificial sea water (425 mM NaCl, 9 mM KCl, 9.3 mM CaCl₂, 25.5 mM MgSO₄, 23 mM MgCl₂, 2 mM NaHCO₃, pH 8), with 20% (v/v) Arab medium crude oil. Separately, artificial sea water was sonicated with 20% (v/v) Arab medium crude oil in the absence of added surfactant. In the absence of surfactant, sonication of the crude oil with artificial sea water yielded an intractable semi-solid sludge. In the presence of peptide SEQ ID NO: 4, a low viscosity emulsion was obtained. In the presence of peptides SEQ ID NOs: 5 or 8, a slightly more viscous but still flowable emulsion was obtained. Sizing of the emulsions was not possible due to the strong colour of the crude oil. On addition of 0.1% (w/v) poly(hexamethylbiguanide), the emulsion formed with peptide SEQ ID NO: 4 rapidly flocculated, then released a clear aqueous phase and flowable crude oil. The example demonstrates that peptides as described in this specification may be useful in increasing the extraction of crude oil in the presence of brine, and that the resulting emulsion can be broken by the addition of a suitable polymer.

### Example 10

### Preparation of Emulsions of Canola Oil in Phosphate-Buffered Saline Using peptides SEQ ID NOs: 5 and 8

Emulsions were prepared by sonicating aqueous solutions of 2.5 mM SEQ ID NOs: 5 or 8 in phosphate-buffered saline (10 mM phosphate, 150 mM NaCl, pH 7.3-7.5) with 20% (v/v) food-grade canola oil. Samples of the emulsions were diluted in phosphate-buffered saline and the droplet size distribution and zeta potential were determined using a Zetasizer Nano ZS. The Sauter mean diameter and zeta potential are given in Table 14. The results show that emulsions stable in the presence of physiological salt solutions, similar to those in blood, can be obtained with a food-grade oil, indicating that the peptides may be useful in the preparation of emulsions for parenteral nutrition.

TABLE 14
Effect of peptide sequence on the Sauter mean diameter
and zeta potential of droplets in emulsions prepared in
phosphate-buffered saline with 20% (v/v) canola oil.

| Peptide | d₃₂ (nm) | Zeta potential (mV) |
|---|---|---|
| SEQ ID NO: 5 | 341 | −45.4 |
| SEQ ID NO: 8 | 737 | −41.2 |

### Example 11

### Preparation of Emulsions of Miglyol 812 in Phosphate-Buffered Saline Using Peptides SEQ ID NOs: 4, 5 and 8

Emulsions were prepared by sonicating aqueous solutions of 2.5 mM SEQ ID NOs: 4, 5 or 8 in phosphate-buffered saline (10 mM phosphate, 150 mM NaCl, pH 7.3-7.5) with 20% (v/v) Miglyol 812 (medical-grade medium-chain triglycerides). Samples of the emulsions were diluted in phosphate-buffered saline and the droplet size distribution and zeta potential were determined using a Zetasizer Nano ZS. The Sauter mean diameter and zeta potential are given in Table 15. The results show that emulsions stable in the presence of physiological salt solutions, similar to those in blood, can be obtained with a medical-grade triglyceride, indicating that the peptides may be useful in the preparation of emulsions for parenteral nutrition or delivery of hydrophobic drugs dissolved in a carrier oil.

TABLE 15
Effect of peptide sequence on the Sauter mean diameter
and zeta potential of droplets in emulsions prepared in
phosphate-buffered saline with 20% (v/v) Miglyol 812.

| Peptide | d₃₂ (nm) | Zeta potential (mV) |
|---|---|---|
| SEQ ID NO: 5 | 329 | −30.1 |
| SEQ ID NO: 5 | 264 | −42.5 |
| SEQ ID NO: 8 | 345 | −41.0 |

### Example 12

### Preparation of Emulsions Using Peptide SEQ ID NOs: 6, 7 or 9. Effects of Sodium Nitrate

A series of emulsions was prepared by sonicating aqueous solutions of 1 mM SEQ ID NOs: 6, 7 or 9, 0.0-2.0 M NaNO₃, pH 8.0, with 20% (v/v) methyl oleate. It was found that at 0.5-2.0 M NaNO₃, increasing degrees of coalescence were observed following sonication. Samples of emulsions prepared at 0.0-0.2 M NaNO₃ were diluted in the corresponding salt compositions and the droplet size distribution and zeta potential were determined using a Zetasizer Nano ZS. The Sauter mean diameter and zeta potential at selected salt concentrations are given in Table 16. It can be seen that for these peptides, the resistance to coalescence is less with the mildly chaotropic salt NaNO₃ than with sodium chloride, which is not considered to be a chaotropic salt.

TABLE 16
Effect of NaNO₃ on the Sauter mean diameter and zeta potential
of droplets in emulsions prepared with 1 mM SEQ ID NOs:
6, 7 or 9 pH 8.0 and 20% (v/v) methyl oleate.

| | Salt | | |
|---|---|---|---|
| Peptide | Concentration | d₃₂ (nm) | Zeta potential (mV) |
| SEQ ID NO: 6 | 0 mM (NaNO₃) | 413 | −60.3 |
| SEQ ID NO: 6 | 100 mM (NaNO₃) | 256 | −44.6 |
| SEQ ID NO: 6 | 200 mM (NaNO₃) | 260 | −38.5 |
| SEQ ID NO: 7 | 0 mM (NaNO₃) | 271 | −51.5 |
| SEQ ID NO: 7 | 100 mM (NaNO₃) | 273 | −45.2 |
| SEQ ID NO: 7 | 200 mM (NaNO₃) | 322 | −37.9 |
| SEQ ID NO: 9 | 0 mM (NaNO₃) | 373 | −67.7 |
| SEQ ID NO: 9 | 100 mM (NaNO₃) | 419 | −51.8 |
| SEQ ID NO: 9 | 200 mM (NaNO₃) | 302 | −42.1 |

### Example 13

### Preparation of Emulsions Using Peptide SEQ ID NO: 4. Effects of Sodium or Potassium Chloride

A series of emulsions was prepared by sonicating aqueous solutions of 2 mM SEQ ID NO: 4, 0.0-2.0 M NaCl or KCl, pH 8.0, with 50% (v/v) hexadecane. Samples of the emulsions were diluted in the corresponding salt solution and the droplet size distribution and zeta potential were determined using a Zetasizer Nano ZS. The Sauter mean diameter and zeta potential at selected salt concentrations are given in Table 17. Larger droplet sizes indicate flocculation and/or coalescence of droplets in the emulsion. It can be seen that sensitivity to high salt concentrations is greater with NaCl than KCl for this peptide emulsifier, although at some intermediate salt concentrations a smaller calculated Sauter mean diameter fails to reflect the presence of a population of larger droplets.

TABLE 17
Effect of salts on the Sauter mean diameter
of droplets in emulsions prepared with 1 mM
SEQ ID NO: 4 pH 8.0 and 50% (v/v) hexadecane.

| Salt Concentration | d₃₂ (nm) | Zeta potential (mV) |
|---|---|---|
| 0 mM (KCl) | 750 | −79.5 |
| 100 mM (KCl) | 678 | −53.6 |
| 200 mM (KCl) | 714 | −46.2 |
| 500 mM (KCl) | 598 | −32.3 |
| 1.0M (KCl) | 4204 | −23.0 |
| 2.0M (KCl) | 3167 | −16.2 |
| 100 mM (NaCl) | 1498 | −50.5 |
| 200 mM (NaCl) | 1343 | −44.8 |
| 500 mM (NaCl) | 738 | −32.3 |
| 1.0M (NaCl) | 351 | −19.6 |
| 2.0M (NaCl) | 3316 | −2.4 |

### Example 14

### Preparation of an Emulsion Using Peptide SEQ ID NO: 11. Effects of Ammonium Chloride and Ammonium Sulfate

A series of emulsions was prepared by sonicating aqueous, solutions of 2 mM SEQ ID NO 11, 0.0-2.0 M NH₄Cl or (NH₄)SO₄, pH 8.0, with 20% (v/v) Cropspray 11N. It was found that emulsions prepared at 0.2-2.0 M NH₄Cl or 0.1-1.0 M (NH₄)SO₄ coalesced on standing. However, an emulsion prepared in 2.0 M (NH₄)₂SO₄ was found to be stable to coalescence. Samples of the non-coalesced emulsions were diluted in the corresponding salt solution and the droplet size distribution and zeta potential were determined using a Zetasizer Nano ZS. The Sauter mean diameter and zeta potential at selected salt concentrations are given in Table 18. Larger droplet sizes indicate flocculation and/or coalescence of droplets in the emulsion. On dilution of the emulsion containing 2.0 M (NH₄)₂SO₄ with an equal volume of water, the emulsion coalesced. The result shows that high salt concentrations can in some cases confer coalescence stability on an emulsion, and dilution to give a lower salt concentration can be used as a means of coalescing an emulsion.

TABLE 18
Effect of salts on the Sauter mean diameter and zeta
potential of droplets in emulsions prepared with 2 mM
SEQ ID NO: 11 pH 8.0 and 20% (v/v) Cropspray 11N.

| Salt Concentration | d₃₂ (nm) | Zeta potential (mV) |
|---|---|---|
| 0 mM (NH₄Cl) | 570 | −39.7 |
| 100 mM (NH₄Cl) | 878 | −37.5 |
| 2.0M ((NH₄)₂SO₄) | 4529 | −31.4 |

### Example 15

### Preparation of Emulsions Using Peptide SEQ ID NO: 4. Effects of Sodium Perchlorate or Sodium Thiocyanate

A series of emulsions was prepared by sonicating aqueous solutions of 1 mM SEQ ID NO: 4, 0.0-2.0 M NaClO₄ or NaSCN, pH 8.0, with 20% (v/v) Solvesso 200 ND. It was found that at 1.0-2.0 M NaClO₄ or NaSCN, increasing degrees of coalescence were observed following sonication. Samples of the emulsions prepared at 0.0-0.5 M NaClO₄ or NaSCN were diluted in the corresponding salt solution and the droplet size distribution and zeta potential were determined using a Zetasizer Nano ZS. The Sauter mean diameter and zeta potential at selected salt concentrations are given in Table 19. Larger droplet sizes indicate flocculation and/or coalescence of droplets in the emulsion. It can be seen that for peptide SEQ ID NO: 4, the resistance to coalescence is less with the chaotropic salts NaClO₄ or NaSCN than with NaCl, which is not considered to be a chaotropic salt. Sauter mean diameters were not determined at 0.5 M NaClO₄ or NaSCN due to the high polydispersity of the emulsions.

TABLE 19
Effect of salts on the Sauter mean diameter of
droplets in emulsions prepared with 1 mM SEQ ID
NO: 4 pH 8.0 and 20% (v/v) Solvesso 200 ND.

| Salt Concentration | d₃₂ (nm) | Zeta potential (mV) |
|---|---|---|
| 0 mM (NaClO₄) | 674 | −64.1 |
| 100 mM (NaClO₄) | 696 | −49.5 |
| 200 mM (NaClO₄) | 1208 | −38.9 |
| 500 mM (NaClO₄) | n.d. | −7.04 |
| 100 mM (NaSCN) | 917 | −50.9 |
| 200 mM (NaSCN) | 794 | −39.8 |
| 500 mM (NaSCN) | n.d. | −14.4 |

### Example 16

### Preparation of Emulsions Using Peptide SEQ ID NO: 8 with Mobil 100 Oil. Effects of Sodium or Potassium Chloride

A series of emulsions was prepared by sonicating aqueous solutions of 1 mM SEQ ID NO: 8, 0.0-2.0 M NaCl or KCl, pH 8.0, with 20% (v/v) Mobil 100 (severely treated base oil). Samples of the emulsions were diluted in the corresponding salt compositions and the droplet size distribution and zeta potential were determined using a Zetasizer Nano ZS. The Sauter mean diameter and zeta potential at selected salt concentrations are given in Table 20. Larger droplet sizes indicate flocculation and/or coalescence of droplets in the emulsion. It can be seen that sensitivity to high salt concentrations is similar between NaCl and KCl for this peptide emulsifier.

TABLE 20
Effect of salts on the Sauter mean diameter and zeta
potential of droplets in emulsions prepared with
1 mM SEQ ID NO: 8 pH 8.0 and 20% (v/v) Mobil 100.

| Salt Concentration | d₃₂ (nm) | Zeta potential (mV) |
|---|---|---|
| 0 mM (KCl) | 2082 | −107.0 |
| 100 mM (KCl) | 1543 | −64.2 |
| 200 mM (KCl) | 1938 | −48.4 |
| 500 mM (KCl) | 946 | −34.4 |
| 1.0M (KCl) | 895 | −26.2 |
| 2.0M (KCl) | 1637 | −17.2 |
| 100 mM (NaCl) | 981 | −59.4 |
| 200 mM (NaCl) | 861 | −46.7 |
| 500 mM (NaCl) | 1608 | −27.7 |
| 1.0M (NaCl) | 1674 | −19.0 |
| 2.0M (NaCl) | 879 | −10.9 |

### Example 17

### Preparation of Emulsions Using Peptide SEQ ID NO: 8 with Jurong 150 Oil. Effects of Sodium or Potassium Chloride

A series of emulsions was prepared by sonicating aqueous solutions of 1 mM SEQ ID NO: 8, 0.0-2.0 M NaCl or KCl, pH 8.0, with 20% (v/v) Jurong 150 (severely treated base oil). Samples of the emulsions were diluted in the corresponding salt compositions and the droplet size distribution and zeta potential were determined using a Zetasizer Nano ZS. The Sauter mean diameter and zeta potential at selected salt concentrations are given in Table 21. Larger droplet sizes indicate flocculation and/or coalescence of droplets in the emulsion. It can be seen that sensitivity to high salt concentrations is similar between NaCl and KCl for this peptide emulsifier.

TABLE 21
Effect of salts on the Sauter mean diameter and zeta
potential of droplets in emulsions prepared with 1
mM SEQ ID NO: 8 pH 8.0 and 20% (v/v) Jurong 150.

| Salt Concentration | d₃₂ (nm) | Zeta potential (mV) |
|---|---|---|
| 0 mM (KCl) | 1251 | −112.0 |
| 100 mM (KCl) | 1022 | −58.1 |
| 200 mM (KCl) | 2334 | −55.8 |
| 500 mM (KCl) | 1113 | −35.6 |
| 1.0M (KCl) | 2701 | −23.4 |
| 2.0M (KCl) | 1596 | −17.1 |
| 100 mM (NaCl) | 1712 | −62.5 |
| 200 mM (NaCl) | 2845 | −44.3 |
| 500 mM (NaCl) | 1561 | −28.0 |
| 1.0M (NaCl) | 2766 | −17.4 |
| 2.0M (NaCl) | 2814 | −9.3 |

### Example 18

### Preparation of Model Pharmaceutical Emulsions in Phosphate-Buffered Saline Using Peptides SEQ ID NOs: 4, 5 and 8

A series of emulsions was prepared by sonicating aqueous solutions of 2.5 mM SEQ ID NOs: 4, 5 and 8 in phosphate-buffered saline with 50% (v/v) glyceryl trioctanoate containing 0.1% (w/v) paclitaxel from Taxus brevifolia. Samples of the emulsions were diluted in the phosphate-buffered saline and the droplet size distribution and zeta potential were determined using a Zetasizer Nano ZS. The Sauter mean diameter and zeta potential are given in Table 22. It can be seen that droplets of less than 1 μm size can be prepared directly in phosphate-buffered saline using these peptide emulsifiers. Such droplets may serve as carriers of the anti-cancer active paclitaxel, for intravenous delivery.

TABLE 22
Properties of emulsions prepared with 2.5 mM SEQ ID NOs:
4, 5 or 8 in phosphate-buffered saline with 50% (v/v) glyceryl
trioctanoate containing 0.1% (w/v) paclitaxel.

| Peptide | d₃₂ (nm) | Zeta potential (mV) |
|---|---|---|
| SEQ ID NO: 4 | 741 | −30.0 |
| SEQ ID NO: 5 | 663 | −39.0 |
| SEQ ID NO: 8 | 523 | −41.5 |

### REFERENCES

Binks, B. P. 1998. Modern Aspects of Emulsion Science. Cambridge, Royal Society of Chemistry.
Collins, K. D. 2004: Ions from the Hofmeister series and osmolytes: effects on proteins in solution and in the crystallization process. Methods 34, 300-311.
Jones, D. B. and Middelberg A. P. J. 2002. Mechanical properties of interfacially adsorbed peptide networks, Langmuir, 18, 10357-10362.
Lin, B., McCormick, A. V., Davis, H. T. and Strey, R. 2005. Solubility of sodium soaps in aqueous salt solutions. J. Colloid Interface Sci. 291, 543-549.
Marcus, Y. 1986. The Hydration Entropies of Ions and Their Effects on the Structure of Water. Journal of the Chemical Society-Faraday Transactions 182, 233-242.
Marcus, Y. 1987. The Thermodynamics of Solvation of Ions .2. the Enthalpy of Hydration at 298.15-K. Journal of the Chemical Society-Faraday Transactions 183, 339-349.
Marcus, Y. 1991. Thermodynamics of Solvation of Ions .5. Gibbs Free-Energy of Hydration at 298.15-K. Journal of the Chemical Society-Faraday Transactions 87, 2995-2999.
Zhang, Z. C. C., Dery, M., Zhang, S. G. and Steichen, D. 2004. New process for the production of branched-chain fatty acids. J. Surfactants Deterg. 7, 211-215.

1. A method of stabilizing an emulsion against flocculation or coalescence, said method comprising forming the emulsion in the presence of at least one peptide emulsifier, said at least one peptide emulsifier comprising one or more amino acid residues with a side chain carboxylate groups.

2. The method according to claim 1, where the peptide emulsifier is a β-strand peptide.

3. The method according to claim 1, where the peptide emulsifier is a α-helical peptide.

4. The method according to claim 1, where the peptide emulsifier has a net negative charge.

5. The method according to claim 1, where the peptide emulsifier has a net positive charge.

6. The method according to claim 1, where the peptide emulsifier is a zwitterionic peptide.

7. The method according to claim 1, wherein the peptide emulsifier has more than one amino acid residue with a side chain carboxylate group.

8. The method according to claim 7, wherein the peptide emulsifier has 2 to 10 amino acid residues with side chain carboxylate groups.

9. The method according to claim 3, wherein the one or more carboxylate groups are oriented around the liquid-liquid interface.

10. The method according to claim 1 wherein the at least one peptide emulsifier is a mixture of peptide emulsifiers.

11. The method according to claim 1, wherein the emulsion is an oil-in-water emulsion.

12. The method according to claim 1, wherein the emulsion is stable in the presence of salt.

13. The method according to claim 12, wherein the salt is present when forming the emulsion.

14. The method according to claim 12, wherein the salt is added to the emulsion after formulation.

15. A method of making an emulsion resistant to flocculation or coalescence comprising:
mixing an aqueous phase and an oil phase together in the presence of at least one peptide emulsifier comprising one or more amino acid residues with a side chain carboxylate group.

16. A salt-resistant emulsion comprising at least one peptide emulsifier comprising one or more amino acid residues with a side chain carboxylate group.

17. A peptide having one of the following sequences:
SEQ ID NO: 2
Ac-LEELADS LEELAEQ VEELLSA-CONH₂
SEQ ID NO: 3
Ac-EISALEA EISALEA EISALEA-CONH₂
SEQ ID NO: 4
Ac-AISELEA EISALEA EIESLAA-CONH₂
SEQ ID NO: 5
Ac-AIESLAE SIEELAE AISELAA-CONH₂
SEQ ID NO: 6
H₂N-P LAEIADS LAEIAEQ VAELIEA VED-CO₂H
SEQ ID NO: 7
H₂N-P LEAIADS LEAIAEQ VEALIEA VAD-CO₂H
SEQ ID NO: 8
H₂N-PG IAELEAE LSAVAEA LEAILAE LD-CO₂H
SEQ ID NO: 9
Ac-LAELESL LAELEAL VAELLSA-CONH₂.


SEQUENCE LISTING (11 sequences)

SEQ ID NO: 1 - protein, 21 aa, Artificial Sequence
  Synthetic
MEELADSLEELARQVEELESA

SEQ ID NO: 2 - protein, 21 aa, Artificial Sequence
  Synthetic
LEELADSLEELAEQVEELLSA

SEQ ID NO: 3 - protein, 21 aa, Artificial Sequence
  Synthetic
EISALEAEISALEAEISALEA

SEQ ID NO: 4 - protein, 21 aa, Artificial Sequence
  Synthetic
AISELEAEISALEAEIESLAA

SEQ ID NO: 5 - protein, 21 aa, Artificial Sequence
  Synthetic
AIESLAESIEELAEAISELAA

SEQ ID NO: 6 - protein, 25 aa, Artificial Sequence
  Synthetic
PLAEIDSALAEIEAQVAELIAAVED

SEQ ID NO: 7 - protein, 25 aa, Artificial Sequence
  Synthetic
PLEAIADSLEAIAEQVEALIEAVAD

SEQ ID NO: 8 - protein, 25 aa, Artificial Sequence
  Synthetic
PGIAELEAELSAVEAELEAILAELD

SEQ ID NO: 9 - protein, 21 aa, Artificial Sequence
  Synthetic
LAELESLLAELEALVAELLSA

SEQ ID NO: 10 - protein, 9 aa, Artificial Sequence
  Synthetic
PDFDFDFDP

SEQ ID NO: 11 - protein, 9 aa, Artificial Sequence
  Synthetic
PEFEFEFEP